SEQUENCE LISTING (21 sequences)

SEQ ID NO: 1 - protein, 10 aa, Hepatitis C virus
DEMEECASHL

SEQ ID NO: 2 - protein, 265 aa, Artificial Sequence
  A mutant sequence from Aequorea victoria
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRSRAQASNSAVDGTAGPGSTGSR

SEQ ID NO: 3 - DNA, 798 bp, Artificial Sequence
  A mutant sequence from Aequorea victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatctagataa

SEQ ID NO: 4 - protein, 519 aa, Homo sapiens
MLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGYSRVGAAGRFEQT

SEQ ID NO: 5 - DNA, 1560 bp, Homo sapiens
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatga

SEQ ID NO: 6 - DNA, 59 bp, Artificial Sequence
  Primer
gtaaggatgagatggaagagtgcgcctcacacctcctgctgctgctgctgctgctgggc

SEQ ID NO: 7 - DNA, 26 bp, Artificial Sequence
  Primer
gtaagttgttgttaacttgtttattg

SEQ ID NO: 8 - DNA, 37 bp, Artificial Sequence
  Primer
ataaagcttccaccatggcgcccatcacggcgtacgc

SEQ ID NO: 9 - DNA, 32 bp, Artificial Sequence
  Primer
gcgggatccttaagagcactcttccatctcat

SEQ ID NO: 10 - DNA, 27 bp, Artificial Sequence
  Primer
ttgaaaggctccgcggggggtccgctg

SEQ ID NO: 11 - DNA, 27 bp, Artificial Sequence
  Primer
cagcggaccccccgcggagcctttcaa

SEQ ID NO: 12 - DNA, 40 bp, Artificial Sequence
  Primer
acatagatatcccaccatgctgctgctgctgctgctgctg

SEQ ID NO: 13 - DNA, 35 bp, Artificial Sequence
  Primer
acgtagatatctcatgtctgctcgaagcggccggc

SEQ ID NO: 14 - DNA, 84 bp, Artificial Sequence
  Primer
gatatacatatgggttctgttgttattgttggtagaattattttatctggtagtggtagtatcacggcgtacgcccagcagacg

SEQ ID NO: 15 - DNA, 39 bp, Artificial Sequence
  Primer
ctcagcgaattctcacgtgacgacctccaggtcggccga

SEQ ID NO: 16 - DNA, 62 bp, Artificial Sequence
  Primer
agtatgccgtattgctaatacgactcactatagggatggtgagcaagggcgaggagctgttc

SEQ ID NO: 17 - DNA, 21 bp, Artificial Sequence
  Primer
gccgatttcggcctattggtt

SEQ ID NO: 18 - DNA, 26 bp, Artificial Sequence
  Primer
ccaccgccaccatggtgagcaagggc

SEQ ID NO: 19 - DNA, 21 bp, Artificial Sequence
  Primer
tcatgtctgctcgaagcggcc

SEQ ID NO: 20 - DNA, 39 bp, Artificial Sequence
  Primer
atgctggattcccaccatggcgcccatcacggcgtacgc

SEQ ID NO: 21 - DNA, 34 bp, Artificial Sequence
  Primer
atgcgggattcttaagagcactcttccatctcat

(19) United States
(12) Patent Application Publication
(10) Pub. No.: US 2005/0053921 A1
(43) Pub. Date: Mar. 10, 2005

(54) Systems for screening anti-hepatitis drugs

(76) Inventors: Tsu-An Hsu, Taipei (TW); Jin-Ching Lee, Taichung (TW)

Correspondence Address: FISH & RICHARDSON PC, 225 FRANKLIN ST, BOSTON, MA, 02110, US

(21) Appl. No.: 10/837,874

(22) Filed: May 3, 2004

Related U.S. Application Data
(60) Provisional application No. 60/467,734, filed on May 2, 2003.

(51) Int. Cl. C12Q 1/70; C12N 9/48

(52) U.S. Cl. 435/5; 435/212

(57) ABSTRACT

Polypeptides each having a target sequence that contains a recognition site of a protease and is flanked by a first heterologous sequence and a second heterologous sequence. Disclosed are nucleic acids encoding the polypeptides, and vectors and host cells having the nucleic acids. Also disclosed are systems containing the polypeptides or the nucleic acids and use of the systems.

### RELATED APPLICATION

This application claims priority to U.S. Provisional Application Ser. No. 60/467,734, filed May 2, 2003, the contents of which are incorporated herein by reference.

### BACKGROUND

Hepatitis can be caused by many infection agents, including hepatitis A, B, C, and D viruses and GB virus. Viral hepatitis is the major cause of liver disease. Take hepatitis C for example, it is estimated to affect 170 million people worldwide. Most patients with liver damage resulting from hepatitis C may develop chronic liver diseases, such as cirrhosis and hepatocellular carcinoma. Hepatitis C can be treated with interferon α. However, only about 50% hepatitis C patients are responsive to the treatment. Other drawbacks to interferon α therapy include significant side effects, high costs, and poor responsiveness to hepatitis C virus 1b, the most common genotype in the United States. New therapies have been vigorously sought. Although several drug candidates are now being evaluated, the progress is rather slow due to a lack of appropriate screening systems. Thus, there is a need for a reliable system and method for identifying drugs for treating hepatitis C and other viral hepatitis.

### SUMMARY

This invention relates to a fusion polypeptide that, after being proteolyzed, generates a detectable heterologous reporter. As proteases are essential for processing structural and none-structural proteins of a number of viruses, a system having the fusion polypeptide can be used to screen for drugs for treating an infection by a virus. It is also useful in determining the protease activity of a biological preparation or identifying a composition that inhibits the activity of a protease.

One aspect of the invention features a polypeptide having a target sequence that contains a protease recognition site that is flanked by a first heterologous sequence and a second heterologous sequence. The first heterologous sequence and the second heterologous sequence can be two reporters, such as Enhanced Green Fluorescent Protein (EGFP or EG) and Secreted Alkaline Phosphatase (SEAP). The polypeptide sequences and the corresponding open reading frames (ORF) of EGFP and SEAP are listed below.

EGFP Polypeptide Sequence:

Mvskgeelftgvvpilveldgdvnghkfsvsgegegdatygktlkficttgklpvpwptlvttltygvqcfsrypdhmkqh
dffksampegyvqertiffkddgnyktraevkfegdtlvnrielkgidfkedgnilghkleynynshnvyimadkqkngik
vnfkirhniedgsvqladhyqqntpigdgpvllpdnhylstqsalskdpnekrdhmvllefvtaagitlgmdelyksglrsra
qasnsavdgtagpgstgsr

EGFP ORF:

atggtgagcaagggcgaggagctgttcaccggggtggtgcccatcctggtcgagctggacggcgacgtaaacggccacaag
ttcagcgtgtccggcgagggcgagggcgatgccacctacggcaagctgaccctgaagttcatctgcaccaccggcaagctgc
ccgtgccctggcccaccctcgtgaccaccctgacctacggcgtgcagtgcttcagccgctaccccgaccacatgaagcagcac
gacttcttcaagtccgccatgcccgaaggctacgtccaggagcgcaccatcttcttcaaggacgacggcaactacaagacccg
cgccgaggtgaagttcgagggcgacaccctggtgaaccgcatcgagctgaagggcatcgacttcaaggaggacggcaacat
cctggggcacaagctggagtacaactacaacagccacaacgtctatatcatggccgacaagcagaagaacggcatcaaggtg
aacttcaagatccgccacaacatcgaggacggcagcgtgcagctcgccgaccactaccagcagaacacccccatcggcgac
ggccccgtgctgctgcccgacaaccactacctgagcacccagtccgccctgagcaaagaccccaacgagaagcgcgatcac
atggtcctgctggagttcgtgaccgccgccgggatcactctcggcatggacgagctgtacaagtccggactcagatctcgagct
caagcttcgaattctgcagtcgacggtaccgcgggcccgggatccaccggatctagataa

SEAP Polypeptide Sequence:

mlllllllglrlqlslgiipveeenpdfwnreaaealgaakklqpaqtaaknliiflgdgmgvstvtaarilkgqkkdklgpeipl
amdrfpyvalsktynvdkhvpdsgatataylcgvkgnfqtiglsaaarfnqcnttrgnevisvnmrakkagksvgvvtttrv
qhaspagtyahtvnrnwysdadvpasarqegcqdiatqlisnmdidvilgggrkymfrmgtpdpeypddysqggtrldg
knlvqewlakrqgaryvwnrtelmqasldpsvthlmglfepgdmkyeihrdstldpslmemteaalrllsrnprgfflfveg
gridhghhesrayraltetimfddaieragqltseedtlslvtadhshvfsfggyplrgssifglapgkardrkaytvllygngpg
yvlkdgarpdvtesesgspeyrqqsavpldeethagedvavfargpqahlvhgvqeqtfiahvmafaaclepytacdlapp
agttdaahpgysrvgaagrfeqt

SEAP ORF:

atgctgctgctgctgctgctgctgggcctgaggctacagctctccctgggcatcatcccagttgaggaggagaacccggacttct
ggaaccgcgaggcagccgaggccctgggtgccgccaagaagctgcagcctgcacagacagccgccaagaacctcatcatct
tcctgggcgatgggatgggggtgtctacggtgacagctgccaggatcctaaaagggcagaagaaggacaaactggggcctg
agatacccctggccatggaccgcttcccatatgtggctctgtccaagacatacaatgtagacaaacatgtgccagacagtggag
ccacagccacggcctacctgtgcggggtcaagggcaacttccagaccattggcttgagtgcagccgcccgctttaaccagtgc
aacacgacacgcggcaacgaggtcatctccgtgatgaatcgggccaagaaagcagggaagtcagtgggagtggtaaccacc
acacgagtgcagcacgcctcgccagccggcacctacgcccacacggtgaaccgcaactggtactcggacgccgacgtgcct
gcctcggcccgccaggaggggtgccaggacatcgctacgcagctcatctccaacatggacattgacgtgatcctaggtggag
gccgaaagtacatgtttcgcatgggaaccccagaccctgagtacccagatgactacagccaaggtgggaccaggctggacgg
gaagaatctggtgcaggaatggctggcgaagcgccagggtgcccggtatgtgtggaaccgcactgagctcatgcaggcttcc
ctggacccgtctgtgacccatctcatgggtctctttgagcctggagacatgaaatacgagatccaccgagactccacactggacc
cctccctgatggagatgacagaggctgccctgcgcctgctgagcaggaacccccgcggcttcttcctcttcgtggagggtggtc
gcatcgaccatggtcatcatgaaagcagggcttaccgggcactgactgagacgatcatgttcgacgacgccattgagagggcg
ggccagctcaccagcgaggaggacacgctgagcctcgtcactgccgaccactcccacgtcttctccttcggaggctaccccct
gcgagggagctccatcttcgggctggcccctggcaaggcccgggacaggaaggcctacacggtcctcctatacggaaacggt
ccaggctatgtgctcaaggacggcgcccggccggatgttaccgagagcgagagcgggagccccgagtatcggcagcagtca
gcagtgcccctggacgaagagacccacgcaggcgaggacgtggcggtgttcgcgcgcggcccgcaggcgcacctggttca
cggcgtgcaggagcagaccttcatagcgcacgtcatggccttcgccgcctgcctggagccctacaccgcctgcgacctggcg
ccccccgccggcaccaccgacgccgcgcacccgggttactctagagtcggggcggccggccgcttcgagcagacatga

In one embodiment, the protease is a viral protease, such as that of hepatitis virus. In one example, the protease is a hepatitis C virus NS3/4A protease, which recognizes the target sequence DEMEEC-ASHL (Δ4AB, SEQ ID NO: 1) and hydrolyzes the peptide bond between resides C and A (i.e., the cleavage site).

This invention also features an isolated nucleic acid having a sequence that encodes the above-described polypeptide. An “isolated nucleic acid” refers to a nucleic acid the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. The nucleic acid of this invention can be used to express the polypeptide of this invention. For this purpose, one can operatively link the nucleic acid to suitable regulatory sequences to generate an expression vector.

A vector refers to a nucleic acid molecule capable of transporting another nucleic acid to which it has been linked, and also capable of autonomous replication or integration into a host DNA. Examples include a plasmid, cosmid, and viral vector. A vector of this invention includes a nucleic acid in a form suitable for expression of the nucleic acid in a host cell. Preferably, the vector includes one or more regulatory sequences operatively linked to the nucleic acid sequence to be expressed. Examples of a regulatory sequence include promoters, enhancers, and other expression control elements (e.g., polyadenylation signals). Regulatory sequences also include those that direct constitutive expression of a nucleotide sequence, as well as tissue-specific regulatory and/or inducible sequences. The design of such an expression vector is based on considerations including the choice of the host cell to be transformed and the desired expression level. An expression vector can be introduced into host cells to produce the polypeptide of this invention. This invention also includes a host cell that contains the above-described nucleic acid. The host cell can be a bacterial cell, a yeast cell, an insect cell, a plant cell, and a mammalian cell.

To produce a polypeptide of this invention, one can place a host cell in a culture under conditions permitting expression of a polypeptide encoded by a nucleic acid described above, and isolate the polypeptide from the culture. Alternatively, the nucleic acid of this invention can be transcribed and translated in vitro, for example, using T7 promoter regulatory sequences and T7 polymerase.

In another aspect, the invention features a system, suitable for screening inhibitors of a protease, that (1) contains the above-described polypeptide or nucleic acid encoding it and (2) a protease or a nucleic acid encoding the protease. This protease recognizes and proteolyzes the target sequence, thereby producing a proteolytic fragment that contains the second heterologous sequence (e.g., SEAP) and is free of the first heterologous sequence (e.g., EGFP). For example, the system can be a cell expressing the polypeptide. In this system, the proteolytic fragment is secreted out of the cell. To make such a cell, one can express the polypeptide and the protease by introducing nucleic acids encoding them into a suitable host cell. Examples of suitable cells include COS-7, AVA5, and 293FT cells. The system can also be a cell-free system (e.g., a TNT system) or a cell in an animal.

The just-described system is useful in identifying an inhibitor of a protease. More specifically, one can introduce a test composition (e.g., a naturally occurred or synthetic compound, or a mixture of such compounds) to a first system and determine a first level or activity of the proteolytic fragment. The composition is determined to be an inhibitor of the protease if the first level or activity is lower than a second level or activity determined in the same manner on an identical system to which the composition is not introduced. The inhibitor thus identified can be used to treat disorders that are associated with high protease levels, such as apoptosis and viral infection (both of which require proteolysis). Accordingly, the just-described system is also useful in identifying a composition for treating an infection by a virus, including HIV and hepatitis virus, e.g., hepatitis virus C. For this purpose, one should select the protease and the target sequence that are specific for the virus of interest.

Also within the scope of this invention is a system that contains the above-described polypeptide or nucleic acid. One can use this system to determine a protease activity of a biological preparation or the ability of the preparation to induce protease activity of the system. More specifically, one can do so by (i) introducing a biological preparation to a first system, and (ii) measuring a first level or activity of a proteolytic fragment that contains the second heterologous sequence and is free of the first heterologous sequence. The preparation is determined to have a protease activity or an ability to induce protease activity of the system if the first level or activity is higher than a second level or activity determined in the same manner on an identical system to which the biological preparation is not introduced.

The details of one or more embodiments of the invention are set forth in the accompanying description below. Other advantages, features, and objects of the invention will be apparent from the detailed description and the claims.

### DETAILED DESCRIPTION

This invention relates to a fusion polypeptide having a protease recognition site and a screening system having the fusion polypeptide. They can be used to identify anti-viral hepatitis drugs.

A fusion polypeptide of this invention includes a target sequence that (1) contains a protease recognition site and (2) is flanked by a first heterologous sequence and a second heterologous sequence on both sides. The protease recognition site can be that of any protease of interest. In one example, the target sequence contains a site that is recognized by the HCV Ns3/4A protease, e.g., SEQ ID NO: 1. This target sequence, as well as the fusion polypeptide, is therefore a substrate of the protease.

A polypeptide of this invention can be produced by using an expression vector that contains an isolated nucleic acid of this invention. The vector can be designed for expression of the polypeptide in prokaryotic or eukaryotic cells, such as bacterial cells (e.g., E. coli), yeast cells, insect cells, plant cells, and mammalian cells. Suitable host cells are discussed in Goeddel, (1990) Gene Expression Technology: Methods in Enzymology 185, Academic Press, San Diego, Calif. Expression of the polypeptide can be carried out with vectors containing constitutive or inducible promoters directing the expression. Fusing a tag to the amino or carboxyl terminus of a truncated glucanase facilitates purification of soluble polypeptide. Examples of a tag include multiple histidines, glutathione S-transferase (GST), maltose E binding protein, protein A, and suitable peptide epitopes, e.g., HA, Myc, and FLAG.

A vector can be introduced into host cells via conventional transformation or transfection techniques, such as calcium phosphate or calcium chloride co-precipitation, DEAE-dextran-mediated transfection, lipofection, or electroporation. After being transformed or transfected with a vector of this invention, a host cell can be cultured in a medium to express a polypeptide. The expressed polypeptide can then be isolated from the host cell or from the culture medium using standard techniques.

If an expressed polypeptide is fused to one of the tags described above, the polypeptide can be easily purified from a clarified cell lysate or culture medium with an appropriate affinity column, e.g., Ni²⁺ NTA resin for hexa-histidine, glutathione agarose for GST, amylose resin for maltose binding protein, chitin resin for chitin binding domain, and antibody affinity columns for epitope tagged proteins. The polypeptide can be eluted from the affinity column, or if appropriate, cleaved from the column with a site-specific protease. If the polypeptide is not tagged for purification, routine methods in the art can be used to develop procedures to isolate it from cell lysates or the media. See, e.g., Scopes, R K (1994) Protein Purification: Principles and Practice, 3rd ed., New York: Springer-Verlag.

The just-described fusion polypeptide can be used in a system of this invention to identify protease activity of a biological preparation or a composition that inhibits the activity of a protease. Preferably, the system is a cell-based system. The first and second heterologous sequences (e.g., EGFP and SEAP, respectively) serve as reporters. Among them, the first heterologous sequence is a cytosol protein (e.g., EGFP or its functional derivatives), which does not have a signal sequence. It anchors the fusion polypeptide in the cytoplasm. In addition, it allows one to determine whether the fusion polypeptide is present in a system. In contrast, the second heterologous sequence (e.g., SEAP or its functional derivatives) has a signal sequence. After being separated from the rest of the fusion polypeptide by proteolysis, it crosses the plasma membrane, enters the secretory pathway, and translocates outside the cell. One can evaluate its amount by measuring the extracellular reporter activity using standard methods.

A “functional derivative” of a reporter refers to a polypeptide derived from the reporter, including a fusion protein, point mutation, insertion, deletion, or truncation of the reporter. It retains substantially the reporter activity (e.g., enzymatic activity or fluorescence emitting ability) and subcellular location of the reporter.

To screen for composition that inhibits the activity of a protease, one can introduce into the above-described system a protease and a fusion polypeptide containing the cleavage site of the protease. The protease and fusion polypeptide can be introduced as enriched or purified recombinant proteins. Alternatively, they can be introduced in the system by expressing nucleic acids encoding them. When using the system in screening for inhibitors of a viral protease, the protease can be introduced into a cell-based system by infecting the cell with the corresponding virus in addition to the two just-described approaches.

As described in the Examples below, in a cell co-expressing EG(Δ4AB)SEAP and NS3/4A protease, the protease recognized and hydrolyzed the specific cleavage site within Δ4AB to separate EGFP from SEAP. The removal of EGFP resulted in the exposure of the signal peptide in SEAP, which targeted SEAP into the secretory pathway and, finally, into the culture medium. The SEAP activity in the medium is positively correlated with the protease activity and, in turn, negatively correlated with efficacy of a protease inhibitor. In this system, no preparation for cell lysate is needed. As SEAP activity of many samples can be readily evaluated in a parallel fashion, the system of this invention is suitable for high throughput screening.

Conventional screening systems and methods in general are limited to compounds that directly bind to a protease and inhibit its enzymatic activity. In contrast, the above-described system and method allow one to identify compositions that inhibit a protease activity via various mechanisms, such as repressing the expression of subunits or cofactors of a protease or the interaction between the subunits or cofactors. Thus, one can use them to identify new protease-inhibiting compositions.

Test compositions to be screened can be obtained using any of the numerous approaches in combinatorial library methods known in the art. Such libraries include: peptide libraries, peptoid libraries (libraries of molecules having the functionalities of peptides, but with a novel, non-peptide backbone that is resistant to enzymatic degradation), spatially addressable parallel solid phase or solution phase libraries, synthetic libraries obtained by deconvolution or affinity chromatography selection, the “one-bead one-compound” libraries, and antibody libraries. See, e.g., Zuckermann et al. (1994) J. Med. Chem. 37, 2678-85; Lam (1997) Anticancer Drug Des. 12, 145; Lam et al. (1991) Nature 354, 82; Houghten et al. (1991) Nature 354, 84; and Songyang et al. (1993) Cell 72, 767.

Examples of methods for the synthesis of molecular libraries can be found in the art, for example, in: DeWitt et al. (1993) Proc. Natl. Acad. Sci. USA 90, 6909; Erb et al. (1994) Proc. Natl. Acad. Sci. USA 91, 11422; Zuckermann et al. (1994) J. Med. Chem. 37, 2678; Cho et al. (1993) Science 261, 1303; Carrell et al. (1994) Angew. Chem. Int. Ed. Engl. 33, 2059; Carell et al. (1994) Angew. Chem. Int. Ed. Engl. 33, 2061; and Gallop et al. (1994) J. Med. Chem. 37,1233. Libraries of compounds may be presented in solution (e.g., Houghten (1992) Biotechniques 13, 412-421), or on beads (Lam (1991) Nature 354, 82-84), chips (Fodor (1993) Nature 364, 555-556), bacteria (U.S. Pat. No. 5,223,409), spores (U.S. Pat. No. 5,223,409), plasmids (Cull et al. (1992) Proc. Natl. Acad. Sci. USA 89, 1865-1869), or phages (Scott and Smith (1990) Science 249, 386-390; Devlin (1990) Science 249, 404-406; Cwirla et al. (1990) Proc. Natl. Acad. Sci. USA 87, 6378-6382; Felici (1991) J. Mol. Biol. 222, 301-310; and U.S. Pat. No. 5,223,409).

Also within the scope of this invention are pharmaceutical compositions that contain the above-descried active agents and a pharmaceutically acceptable carrier for treating an infection with a virus and a method of using such a composition in an effective amount to treat patents in need thereof. The term “treating” is defined as administration of a composition to a subject, who has a viral infection, with the purpose to cure, alleviate, relieve, remedy, prevent, or ameliorate the disorder, the symptom of the disorder, the disease state secondary to the disorder, or the predisposition toward the disorder. An “effective amount” is an amount of the composition that is capable of producing a medically desirable result, e.g., as described above, in a treated subject. The above-described agents can be formulated into dosage forms for different administration routes utilizing conventional methods. For example, such an agent can be formulated in a capsule, a gel seal, or a tablet for oral administration. The pharmaceutical composition can also be administered via the parenteral route. The dosage required depends on the choice of the route of administration; the nature of the formulation; the nature of the subject's illness; the subject's size, weight, surface area, age, and sex; and other drugs being administered. The efficacy of the pharmaceutical composition can be preliminarily evaluated in vitro. For in vivo studies, the composition can be injected into an animal and its effects on the viral infection are then accessed.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

### A Cell-Free System

Plasmid Construction

In all polymerase chain reactions (PCR), pfu polymerase was used to minimize errors (Promega, Madison, Wis.). Construction of pEG(Δ4AB)SEAP was based on pEGFP-C1 plasmid (Clontech, Palo Alto, Calif.). The entire SEAP gene was amplified by PCR from pSEAP2-control plasmid (Clontech). The forward primer, 5′-GTAAGGATGAGATGGAAGAGTGCGCCTCACACCTCCTGCTGCTGCTGCTGCT GCTGGGC-3′ (encoding NS3/4A protease cleavage site, Δ4AB: DEMEECASHL), and the reverse primer, 5′-GTAAGTTGTTGTTAACTTGTTTATTG-3′, were used to amplify SEAP gene. The PCR product was digested with KpnI and inserted into the KpnI site of pEGFP-C1.

To create an NS3/4A expression vector, a DNA fragment encoding NS3/4A was amplified by PCR using HCV cDNA originating from HCV type 1a (pCV-H77) as a DNA template (Yanagi et al., 1997, Proc. Natl. Acad. Sci. USA 94:8738-8743). The forward and reverse primers used were, respectively, 5′-ATAAAGCTTCCACCAT GGCGCCCATCACGGCGTACGC-3′ (containing a Kozak sequence and the ATG translation start codon), and 5′-GCGGGATCCTTAAGAGCACTCTTCCATCTCAT-3′ (containing a translation stop codon). The PCR products were digested with HindIII and BamHI and then inserted into pcDNA3.1(+) (Invitrogen, Carlsbad, Calif.). The serine codon in the catalytic triad of NS3 was replaced with an alanine codon using the oligonucleotides 5′-TTGAAAGGCTCCGCGGGGGGTCCGCTG-3′ and 5′-CAGCGGACCCCCCGCGGAGCCTTTCAA-3′ to generate mNS3/4A, an inactive mutant. Subsequently, expression cassettes containing NS3/4A (wild type) and mNS3/4A (inactive mutant) were subcloned into pEG(Δ4AB)SEAP vector to generate pEG(Δ4AB)SEAP-NS3/4A and pEG(Δ4AB)SEAP-mNS3/4A. The cloned DNA fragments were confirmed by DNA sequencing.

To construct pcDNA-SEAP, the entire SEAP gene was amplified by PCR using pSEAP2-control plasmid as DNA template (Clontech). The forward primer (5′-ACATAGATATCCCACCATGCTGCTGCTGCTGCTGCTGCTG-3′) contained a Kozak sequence and the ATG translation start codon. The reverse primer (5′-ACGTAGATATCTCATGTCTGCTCGAAGCGGCCGGC-3′) contained a translation stop codon. The PCR products were digested with EcoRV and ligated into the EcoRV site of pcDNA3.1(+) (Invitrogen).

Expression and Purification of Single-Chain NS3/4A Protease (scNS3/4A)

A vector encoding a single-chain NS3/4A, pNS4₂₁₋₃₂-GSGS-NS3₃₋₆₃₁, was constructed as described by Taremi et al. 1998, Protein Sci. 7 (1998) 2143-2149. Briefly, PCR was carried out using HCV type 1a (pCV-H77) cDNA clone as a template (Yanagi et al., 1997, Proc. Natl. Acad. Sci. USA 94:8738-8743). The forward primer (5′-GATATACATATGGGTTCTGTTGTTATTGTTGGTAGAATTATTTTATCTGGTAGTGG TAGTATCACGGCGTACGCCCAGCAGACG-3′) contained an NdeI site, the ATG translation initiation codon, amino residues 21-32 of the NS4A cofactor, the GSGS linker, and amino acid residues 3-10 of NS3. The reverse primer (5′-CTCAGCGAATTCTCACGTGACGACCTCCAGGTCGGCCGA-3′) contained the complementary sequences of the EcoRI cloning site, the translation stop codon, and amino acid residues 623-631 of NS3. The PCR products were digested with NdeI and EcoRI and inserted into plasmid pET28a (Novagen, Madison, Wis.). The resulting expression plasmid was used to transform Escherichia coli BL21 (DE3) for protein expression according to standard procedures (Studier et al., 1986, J. Mol. Biol. 189: 113-130. The hexa-histidine-tagged scNS3/4A was purified by nickel-chelated chromatography Taremi et al. 1998, Protein Sci. 7 (1998) 2143-2149.

Protease Assay

In vitro transcription and translation (TNT) was used to produce a substrate protein, EG(Δ4AB)SEAP. A DNA template encoding it was obtained by PCR. The forward primer (5′-AGTATGCCGTATTGCTAATACGACTCACTATAGGGATGGTGAGCAAGGG CGAGGAGCTGTTC-3′) contained the promoter sequence for the T7 RNA polymerase (underlined). The reverse primer (5′-GCCGATTTCGGCCTATTGGTT-3′) contained a translational stop codon. PCR products were transcribed and translated in vitro using a rabbit reticulocyte lysates kit (Promega). To generate ³⁵S-labeled substrates, reactions were carried out at 30° C. for 2 hours in the presence of [³⁵S]methionine and [³⁵S]cysteine (100 μCi/ml, Institute of Isotopes Co., Ltd., Budapest, Hungary). Aliquots of purified single-chain NS3/4A protease were then mixed with the translated EG(Δ4AB)SEAP fusion protein and incubated for 30 minutes at 30° C. The reactions were terminated by mixing with a SDS-PAGE sample buffer and being boiled for 3 minutes. The samples were then analyzed by SDS-PAGE using 4-20% Tris-glycine gel (Invitrogen).

It was found that expression of EGFP or SEAP gene alone yielded prominent band that was close to the expected size of SEAP or EGFP (SEAP: ˜56 kDa; EGFP: ˜30 kDa). The molecular weight of the TNT product of EG(Δ4AB)SEAP fusion protein was close to 87 kDa. When the EGFP-SEAP fusion protein was incubated with scNS3/4A protease, distinct protein bands of approximately 56 and 30 kDa were observed. These results suggest that scNS3/4A efficiently cleaves EG(Δ4AB)SEAP to separate it into two individual proteins: EGFP and SEAP.

### EXAMPLE 2

### Two Systems Based on Transiently Transfected Cells

1. A COS-7 Cell-Based System

COS-7, a monkey kidney cell line, was obtained from the Food Industry Research and Development Institute (Taiwan) and maintained in Dulbecco's modified Eagle's medium (DMEM) supplemented containing 10% fetal bovine serum (FBS, Life Technologies, Grand Island, N.Y., USA).

The expression of the above-describe pEG(Δ4AB)SEAP dual-reporter construct was evaluated by transient transfection of COS-7 cells. More specifically, COS-7 cells were seeded in 24-well plates at a density of 6×10⁴ cells/well in 1 ml of DMEM/10% FBS. After incubation at 37° C. overnight or until cells were approximately 70% confluent, the cells were transfected with various plasmid constructs using the LipofectAMINE 2000 reagent (Life Technologies). As an internal control for transfection efficiency, 0.1 μg pCMV-β-gal (Clontech) plasmid was cotransfected with 0.5 μg of each expression vector for each experiment.

After the cotransfection, culture media and cell lysates were harvested at 12-hour intervals. The SEAP activity in the culture medium and the β-galactosidase activity in the cell lysates were measured using Phospha-Light and Galacto-Star assay kits (Tropix, Foster, Calif., USA), respectively. The respective chemiluminescent intensities were detected by a Top Counter Microplate Scintillation and Luminescence Counter (Packard, Meriden, Conn., USA). The intensities reflected the relative activities of SEAP and galactose. In these experiments, the β-gal activity was used to normalize transfection efficiency according standard techniques. Meanwhile, fluorescence emitted from the green fluorescent protein was examined using a fluorescence microscope (Nikon DIAPHOT 300).

It was found that, at hour 48 post-transfection, many cells in the pEG(Δ4AB)SEAP-transfected cultures emitted strong fluorescent. The intensity of fluorescent from these cells was indistinguishable from that from the pEGFP-C1 transfected cells, indicating that EGFP, when fused to Δ4AB and SEAP, was able to fold properly to emit fluorescence upon excitation. In addition, it was found that the SEAP activity increased in the culture medium of cells transfected with pEG(Δ4AB)SEAP-NS3/4A progressively over time. It was up to approximately 12 times greater than the SEAP activity in culture medium of the cells transfected with pEG(Δ4AB)SEAP-mNS3/4A. This experiment was repeated at least three times, and similar results were obtained. These results indicate that viral NS3/4A activity can be reflected by the activity of SEAP in the culture medium of cells transfected by pEG(Δ4AB)SEAP-NS3/4A.

Western blotting was used to demonstrate that the junction between EGFP and SEAP was indeed cleaved by NS3/4A protease in COS-7 cells. In this experiment, COS-7 cells were transfected with pEGFP-C1, pEG(Δ4AB)SEAP, pEG(Δ4AB)SEAP-NS3/4A, and pEG(Δ4AB)SEAP-mNS3/4A, respectively, according to the method described above. At hour 48 post transfection, cell lysates were prepare and analyzed by Western blot using anti-EGFP antibodies or anti-NS3 antibodies according to standard procedures (Hsu et al., 1994, Protein Express. Purif. 5 (1994) 595-603). Anti-EGFP antibodies and anti-NS3 antibodies were obtained from Clontech and LTK Biolaboratories (Taipei, Taiwan), respectively. Signals were visualized by an ECL Western blotting system (ECL, Amersham Pharmacia Biotech UK, Amersham PLC, Buckinghamshire, England).

It was found that anti-EGFP antibodies recognized a very strong band of approximately 30 kDa, which corresponded to EGFP. This EGFP band was also detected in cells transfected with pEG(Δ4AB)SEAP-NS3/4A. In contrast, no 30 kDa band was observed in cells lacking wild-type NS3/4A or expressing mutant NS3/4A protease. (the expression of NS3/4A proteases was confirmed by Western blot using anti-NS3 antibodies). Further, in the cells expressing the wild-type NS3/4A, the molecular weight of the protein detected by anti-EGFP was clearly smaller than that in the cell expressing mutant NS3/4A. Clearly, NS3/4A protease recognized and cleaved the EG(Δ4AB)SEAP fusion protein efficiently in COS-7 cells.

2. An Ava5 Cell-Based System

Ava5 cells (containing HCV subgenomic replicons) and Huh-7 (human hepatoma cell line) cells were kindly provided by Dr. Charles Rice (Blight et al., 2000, Science 290: 1972-1974). They were maintained in DMEM/10% FBS/1 mg/ml G418 (Geneticin) and DMEM/10% FBS, respectively. Ava5 cells expressed endogenous NS3/4A protease. The above-described dual-reporter construct was transient transfected into the cells in the same manner descried above except that FuGENE 6 (Roche), instead of LipofectamineTM was used. Western blotting was also conducted in the same manner.

The results showed that a distinct band of the size of free EGFP was observed only in Ava5 cells expressing EG(Δ4AB)SEAP fusion protein, indicating that the endogenous NS3/4A protease in Ava5 cells cleaves the fusion protein. When EG(Δ4AB)SEAP was expressed in Huh-7 cells, only minimal amount of cleaved fusion protein was observed. Further, only minimal SEAP activity could be detected in the culture medium of the Huh cells. In contrast, approximately 13 times more SEAP activity was detected in the culture medium of EG(Δ4AB)SEAP-transfected Ava5 cells.

Ava5 cells expressing EG(Δ4AB)SEAP were then tested for the ability to screen for HCV protease inhibitors. More specifically, the cells were treated with IFN-α (Schering-Plough Corp., Kenilworth, N.J., USA) and examined for the SEAP activity in their medium in the manner described above.

Meanwhile, the levels of HCV RNA were determined using standard techniques. Total cellular RNAs were isolated using the High Pure RNA isolation Kit (Roche, Germany). Three micrograms of the total RNA were denatured at 55° C. in 2M formaldehyde/50% formamide for 15 minutes and separated by denaturing agarose gel electrophoresis. The RNA was then transferred to a positively charged nylon membrane, (BrightStar-Plus, Ambion, Austin, Tex., USA) by a vacuum blotter (Vacu. GeneXL, Pharmacia, Mich., USA). A [α-³²P]dCTP-labeled HCV NS5B gene fragment and a human housekeeping gene glyceraldehyde-3-phosphate dehydrogenase (gapdh) gene fragment were generated using the Rediprime II Random Prime Labeling System (Pharmacia). Northern hybridization was performed using the method described in Kevil et al., 1997, Biochem. Biophys. Res. Commun. 238: 277-279. The protein level of NS3 protease was also determined by standard techniques.

It was found that the HCV RNA levels and HCV NS3 protease levels declined as the 100 IU/ml of IFN-α treatment proceeded, especially after 3 days of treatment. In contrast, the RNA levels of gadph remained the same after IFN-α treatment. The SEAP activity in the culture medium was found decreased, correlating closely with the RNA levels. These results indicate that Ava5 cells transfected with EG(Δ4AB)SEAP can be used to screen anti-HCV compounds.

### EXAMPLE 3

### Two Systems Based on Stably Transfected Cells

1. A 293FT Cell-Based System

To make stably transfected cells, plasmid pLenti-EG(Δ4AB)SEAP was generated. The dual-reporter gene EG(Δ4AB)SEAP was amplified by PCR from pEG(Δ4AB)SEAP plasmid described above. The forward and reverse primers used were 5′-CCA CCG CCA CCA TGG TGA GCA AGG GC-3′ and 5′-TCA TGT CTG CTC GAA GCG GCC-3′. PCR products were then inserted into a pLenti6/V5-TOPO plasmid (Invitrogen, Carlsbad, Calif.) to generate pLenti-EG(Δ4AB)SEAP.

Recombinant lentiviral vector was generated by a transient plasmid transfection according to the method described in Dull et al., 1998, J. Virol. 72, 8463-8471. Briefly, 293FT cells (1×10⁶ cells per 10 cm tissue culture dish) were co-transfected with pLenti-EG(Δ4AB)SEAP and ViraPowerTM Packaging Mix using LipofectamineTM 2000 according to manufacturer's instructions. Two days after transfection, lentiviral particles were collected from the conditioned medium, and cellular debris was cleared off by low-speed centrifugation (800 rpm for 15 minutes).

The sequence encoding NS3/4A was cloned into a separate pLenti6/V5-TOPO vector to generate pLenti-NS3/4A. The backbone pLenti6/V5-TOPO plasmid was first modified to replace the blasticidin gene with zeocin gene to generate pLenti-zeo. The sequence encoding NS3/4A was amplified by PCR using HCV cDNA originating from HCV type 1a (pCV-H77) as a template. The forward and reverse primers used were, respectively, 5′-ATGCTGGATTCCCACCAT GGCGCCCATCACGGCGTACGC-3′ (containing a Kozak sequence and the ATG translation start codon) and 5′-ATGCGGGATTCTTAAGAGCACTCTTCCATCTCAT-3′ (containing the trasnlation stop codon). The resultant PCR fragment was digested with BamHI and then inserted into pLenti-zeo vector to form pLenti-NS3/4A.

The just-described pLenti-EG(Δ4AB)SEAP and pLenti-NS3/4A were co-transfected into to 293EBNA cells. As they carried two different selectable marker genes (blasticidin gene and zeocin gene), stable cell lines could be isolated by sequential blasticidin and zeocin selections.

To select stable cell lines, 293EBNA cells were seeded in 24-well plates (6×10⁴ cells per well). After incubation at 37° C. overnight, the cells were transduced with serial dilutions of lentiviral particles in the presence of 6 μg/ml polybrene (Sigma, St. Louis, Mo., USA). After incubation for 24 hours, the virus-containing medium was replaced by a fresh medium. After another 24 hours, the medium was replaced with a conditional medium containing 10 μg/ml blasticidin. After 3 to 4 weeks, two blasticidin-resistant clones were selected and were examined for expression of EG(Δ4AB)SEAP by Western blot analysis and fluorescent microscopy. Positive cell lines thus-identified were designated as 293-EG(Δ4AB)SEAP.

One of the cloned cell lines was further infected with the lentiviral vector encoding NS 3/4A gene. After zeocin selection (250 μg/ml) for 3 to 4 weeks, one zoscine-resistant clone was obtained. This cell line was confirmed by Western blot to express both NS3/4A protease and EG(D4AB)SEAP, and therefore designated as 293P-EG(Δ4AB)SEAP-NS3/4A. It was found that the cells expressed a distinct band corresponding to the size of free EGFP, presumably resulted from the cleavage of the EG(Δ4AB)SEAP by NS3/4A protease. In naïve 293FT cells, no such EGFP band or NS3 band was detected.

SEAP activities in culture media from the stable clones and naïve 293FTwere examined. In this experiment, naive 293FT cells and 293FT stably transfected with EG(Δ4AB)SEAP were used as background and non-specific cleavage controls, respectively. It was found that conditioned culture media of Huh-7, 293-EG(Δ4AB)SEAP, and 293P-EG(Δ4AB)SEAP cells, contained limited SEAP activity. In contrast, SEAP activity in the culture medium from 293P-EG(Δ4AB)SEAP was approximately 50-fold higher than that from the parental Ava5 cells after 72 hours.

The just-described 293P-EG(Δ4AB)SEAP was used in evaluating an NS3/4A inhibitor BILN 2061 (Lamarre et al., 2003, Nature 426, 186-189). More specifically, 293P-EG(Δ4AB)SEAP-NS3/4A cells were seeded in 96-well plates (1.5×10⁴ cells per well) and cultured at 37° C. overnight. The cells were then incubated with various concentrations of BILN 2061 (0, 0.015, 0.06, 0.24, 0.98, 3.9, 15.6, 62.5, 250, and 1000 nM). Two days later, the culture medium in each well was replaced with fresh phenol red-free DMEM/10% FBS containing the same concentration of drugs. After another day, the culture medium was collected and the SEAP activity was measured using a Phospha-Light assay kit (Tropix, Foster City, Calif., USA) according to manufacturer's instruction.

It was found that the EC₅₀ (Effective Concentration in inhibition of 50% of SEAP reporter gene in culture medium) of BILN-2061 was around 2 nM. Further, BILN-2061 did not cause detectable cellular toxicity as measured by MTS assay at concentration up to 10 μM. This result indicates that 293P-EG(Δ4AB)SEAP-NS3/4A cells can be used to screen for NS3/4A inhibitors.

2. An Ava5 Cell-Based System

Huh-7 or Ava5 cells were seeded in 24-well plates at a density of 2×10⁴ cells per well. After incubation at 37° C. overnight, the cells were transduced with serial dilutions of lentiviral particles in the presence of 6 μg/ml polybrene (Sigma, St. Louis, Mo., USA) in the manner described above. After incubation for 24 hours, the virus-containing medium was replaced by a fresh medium and the cells were incubated for another 2 days. The cells were then selected in the presence of 10 μg/ml of blasticidin for 3-4 weeks.

One blasticidin-resistant Huh-7 clone and two blasticidin-resistant Ava5 clones were obtained. Cell lines thus produced were designated as Huh-7-EG(Δ4AB)SEAP and Ava5-G(Δ4AB)SEAP, respectively. The cells were then examined for expression of EG(Δ4AB)SEAP and the amount NS3 protein by Western blot analysis using anti-EGFP antibody and anti-NS3 antibody, respectively. The two Ava5-EG(Δ4AB)SEAP clones (clones 1 and 2) were found to express EG(Δ4AB)SEAP. They also expressed free EGFP, resulting from the cleavage of the EG(Δ4AB)SEAP by NS3/4A protease. In naive Huh-7 and Ava5 cells, neither free EGFP nor NS3 could be detected.

The cells were also evaluated for SEAP activity in culture medium according to the method described above. It was found that the SEAP activities in the media of Ava5-EG(Δ4AB)SEAP clones 1 and 2 were higher than that in the medium of naive Huh-7 or Ava5 cells by 50 (clone 1) and 40 (clone 2) folds, respectively.

Ava5-EG(Δ4AB)SEAP clone 1 was further treated by IFN-α (0-50 units/ml) in the manner described above. They, the cells were examined for cellular NS3/4A protein and HCV RNA levels by western blot and dot blot, respectively. The dot blot was conducted as follows: RNA was extracted using TRIZOL reagent (Invitrogen). Three micrograms of RNA was dissolved in 100 μl of TE buffer (10 mM Tris-HCl, 1 mM EDTA, pH 7.5) and 60 μl of 20×SSC, and 40 μl of 37% formaldehyde stock solution were added. The samples were incubated at 60° C. for 15 min and then applied to pre-wetted nylon membrane using an S&S Minifold® I dot-blot device (Schleicher & Schuell GmbH, Relliehausen, Germany). Each well was washed twice with 6×SSC. Bound RNA was immobilized onto the membrane by UV-cross linking (Stratagene, La Jolla, Calif., USA) under the autocross-link condition, then hybridization and counting were conducted as described (Lee et al., 2003. Anal. Biochem.316, 162-170a). The membrane was probed with NS5B gene fragment of HCV labeled with [γ-³²P]-dCTP by rediprime II random primer labeling system (Amersham Biosciences, Buckinghamshire, England). The blots were visualized by autoradiography and intensities of the blots were measured by densitometry (UVP GDS-8000 BioImaging System, Upland, Calif., USA).

Meanwhile the SEAP activity in the culture medium was determined. It was found all three parameters decreased. The data were analyzed by the SigmaPlot software (SPSS Inc., Chicago, Ill.). The square of correlation constant between the RNA level and SEAP activity was 0.978, suggesting that the SEAP activity positively correlates with the HCV RNA level very well.

The just-described cell line was used to examined several other known anti-HCV compounds, such as an NS3/4A inhibitor, N-tosyl-1-phenylalanine chloromethyl ketone (TPCK) (Berdichevsky et al., 2003, J. Virol. Methods 107, 245-255.), and IFN-γ (Frese et al., 2002, Hepatology 35, 694-703.). More specifically, Ava5-EG(Δ4AB)SEAP cells were treated with TPCK (at 0, 0.1, 0.2, 0.5, 1.5, 4.4, 13.2 and 40 μM) or IFN-γ (0, 0.01, 0.1, 1, 10, and 100 I.U./ml) for 72 hours. It was found that, when the cells were treated with TPCK, the extracellular SEAP activity showed a dose-dependent decrease. At 0.2 μM, TPCK was able to suppress more than 50% of HCV replication. Similarly, the IFN-γ treatment also repressed the extracellular SEAP activity. The EC50 (effective concentration for 50% inhibition) was below 10 IU/ml.

This result indicate that the Ava5-EG(Δ4AB)SEAP cells can be used to screen for NS3/4A inhibitors.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A polypeptide comprising a target sequence that contains a recognition site of a protease, wherein the target sequence is flanked by a first heterologous sequence and a second heterologous sequence.

2. The polypeptide of claim 1, wherein the protease is a viral protease.

3. The polypeptide of claim 2, wherein the viral protease is a protease of hepatitis virus.

4. The polypeptide of claim 3, wherein the hepatitis virus is hepatitis C virus.

5. The polypeptide of claim 4, wherein the target sequence contains SEQ ID NO: 1.

6. The polypeptide of claim 5, wherein the first heterologous sequence and the second heterologous sequence are reporters.

7. The polypeptide of claim 6, wherein the first heterologous sequence and the second heterologous sequence are Enhanced Green Fluorescent Protein and Secreted Alkaline Phosphatase, respectively.

8. The polypeptide of claim 1, wherein the first heterologous sequence and the second heterologous sequence are reporters.

9. The polypeptide of claim 9, wherein the first heterologous sequence and the second heterologous sequence are Enhanced Green Fluorescent Protein and Secreted Alkaline Phosphatase, respectively.

10. A nucleic acid comprising a sequence encoding the polypeptide of claim 1.

11. A vector comprising the nucleic acid of claim 10.

12. A host cell comprising the nucleic acid of claim 10.

13. The host cell of claim 12, wherein the cell is a bacterial cell, a yeast cell, a plant cell, an insect cell, or a mammalian cell.

14. A method of producing a polypeptide, comprising culturing the host cell of claim 12 in a medium under conditions permitting expression of a polypeptide encoded by the nucleic acid, and purifying the polypeptide from the cultured cell or the medium of the cell.

15. A system comprising
a polypeptide having a target sequence or a nucleic encoding the polypeptide, wherein the target sequence contains a protease recognition site sequence and is flanked by a first heterologous sequence and a second heterologous sequence, and
a protease or a nucleic acid encoding the protease,
wherein the protease recognizes and proteolyzes the target sequence, thereby producing a proteolytic fragment that contains the second heterologous sequence and is free of the first heterologous sequence.

16. The system of claim 15, wherein the protease is a viral protease.

17. The system of claim 16, wherein the viral protease is a protease of hepatitis virus.

18. The system of claim 17, wherein the hepatitis virus is hepatitis C virus.

19. The system of claim 18, wherein the target sequence contains SEQ ID NO: 1.

20. The system of claim 19, wherein the first heterologous sequence and the second heterologous sequence are reporters.

21. The system of claim 20, wherein the first heterologous sequence and the second heterologous sequence are Enhanced Green Fluorescent Protein and Secreted Alkaline Phosphatase, respectively.

22. The system of claim 15, wherein the first heterologous sequence and the second heterologous sequence are reporters.

23. The system of claim 22, wherein the first heterologous sequence and the second heterologous sequence are Enhanced Green Fluorescent Protein and Secreted Alkaline Phosphatase, respectively.

24. The system of claim 15, wherein the system is a cell-free system.

25. The system of claim 15, wherein the system is a cell and the proteolytic fragment is secreted out of the cell.

26. The system of claim 25, wherein the cell is a COS-7 cell, an AVA5 cell, or a 293FT cell.

27. The system of claim 25, wherein the protease is a viral protease.

28. The system of claim 27, wherein the viral protease is a protease of hepatitis virus.

29. The system of claim 28, wherein the hepatitis virus is hepatitis C virus.

30. The system of claim 29, wherein the target sequence contains SEQ ID NO: 1.

31. The system of claim 30, wherein the first heterologous sequence and the second heterologous sequence are reporters.

32. The system of claim 31, wherein the first heterologous sequence and the second heterologous sequence are Enhanced Green Fluorescent Protein and Secreted Alkaline Phosphatase, respectively.

33. The system of claim 15, wherein the system is an animal.

34. A method of identifying an inhibitor of a protease, the method comprising:
introducing a composition to a first system of claim 15, and
determining a first level or activity of the proteolytic fragment,
wherein the composition is determined to be an inhibitor of the protease if the first level or activity is lower than a second level or activity determined in the same manner on an identical system into which the composition is not introduced.

35. The method of claim 34, wherein the system is a cell and the proteolytic fragment is secreted out of the cell.

36. A method of identifying a composition for treating an infection by a virus, the method comprising:
introducing a composition to a first system of claim 15, the protease in the system being a virus protease, and
determining a first level or activity of the proteolytic fragment,
wherein the composition is determined to be a candidate for treating the infection if the first level or activity is lower than a second level or activity determined in the same manner on an identical system into which the composition is not introduced.

37. The method of claim 36, wherein the system is a cell and the proteolytic fragment is secreted out of the cell.

38. The method of claim 37, wherein the viral protease is a protease of hepatitis virus.

39. The method of claim 38, wherein the hepatitis virus is hepatitis C virus.

40. The method of claim 39, wherein the target sequence contains SEQ ID NO: 1.

41. A system comprising a polypeptide having a target sequence or a nucleic encoding the polypeptide, wherein the target sequence contains a protease recognition site sequence and is flanked by a first heterologous sequence and a second heterologous sequence.

42. A method of determining a protease activity of a biological preparation, the method comprising:
introducing a biological preparation to a first system of claim 41, and
determining a first level or activity of a proteolytic fragment that contains the second heterologous sequence and is free of the fist heterologous sequence,
wherein the preparation is determined to have a protease activity if the first level or activity is higher than a second level or activity determined in the same manner on an identical system into which the biological preparation is not introduced.
